(19) United States
(12) Patent Application Publication
(10) Pub. No.: US 2007/0157325 A1
(43) Pub. Date: Jul. 5, 2007

(54) PROCESS FOR IDENTIFICATION OF NOVEL DISEASE BIOMARKERS IN MOUSE MODELS OF ALZHEIMER'S DISEASE INCLUDING TRIPLE TRANSGENIC MICE AND PRODUCTS THEREBY

(76) Inventor: Shahriar Mojtahedian, Costa Mesa, CA (US)

Correspondence Address: GREENBERG TRAURIG LLP, 2450 COLORADO AVENUE, SUITE 400E, SANTA MONICA, CA, 90404, US

(21) Appl. No.: 11/618,530

(22) Filed: Dec. 29, 2006

Related U.S. Application Data
(60) Provisional application No. 60/755,320, filed on Dec. 30, 2005.
(60) Provisional application No. 60/789,511, filed on Apr. 4, 2006.

(51) Int. Cl. A01K 67/027; G01N 33/567

(52) U.S. Cl. 800/12; 800/18; 435/7.2

(57) ABSTRACT

The present disclosure includes methods for the identification of novel biomarkers for Alzheimer's disease. The methods involve genomic, proteomic, bioinformatic, immunochemical, and behavioral assays of 3xTg-AD mice.

### RELATED APPLICATIONS

This application claims the Paris Convention Priority of U.S. Provisional Application Ser. No. 60/755,320 filed on Dec. 30, 2005 and U.S. Provisional Application Ser. No. 60/789,511 filed Apr. 4, 2006, which are incorporated by reference in their entirety.

### BACKGROUND OF THE DISCLOSURE

Alzheimer's disease (AD) is a progressive neurodegenerative disorder characterized by global cognitive dysfunction (particularly memory loss), behavior or personality alterations, and impairments in the performance of the activities of daily living. The memory loss exhibited in AD is dependent on the hippocampal system, comprised of the dentate gyrus, cornu ammonis (CA)1-CA3, and rhinal cortices. As the disease progresses, global amnesia, dependent on other cortical areas, debilitates the individual (Hock and Lamb, 2001). AD is characterized by specific neuropathological alterations, including extracellular β-amyloid-containing (Aβ) plaques, intracellular neurofibrillary tangles (NFT) of abnormally phosphorylated tau (τ) protein, and degeneration of the cholinergic neurons in the basal forebrain (Auld et al., 2002). AD is recognized as the most prevalent dementia in mid-to-late life. It affects 7-10% of individuals over the age of 65 and an estimated 40% of persons over the age of 80. It is currently believed that AD affects over 4.5 million Americans and that 100,000 succumb to the disease annually with a projected 22 million individuals worldwide to develop dementia by the year 2025 (Crentsil, 2004).

According to estimates used by the Alzheimer's Association and the National Institute on Aging, the national direct and indirect annual costs of caring for individuals with AD are at least $100 billion (Ernst and Hay, 1994). AD costs American business an estimated $61 billion a year of which $24.6 billion covers AD patient health care and $36.5 billion covers costs related to caregivers of individuals with the disease, including lost productivity, absenteeism, and worker replacement (Koppel, 2002). In addition, approximately half of all nursing home residents have AD or a related disorder with the average cost for nursing home care being $42,000 per year, but exceeding $70,000 per year in some areas of the United States (Rice, 1993). Finally, based on a 2001 report commissioned by the Alzheimer's Association, by 2010 Medicare costs for beneficiaries with AD are expected to increase 54.5% from $31.9 billion in 2000 to $49.3 billion, and Medicaid expenditures on residential dementia are expected to increase 80% from $18.2 billion to $33 billion in 2010 (Alzheimer's Association, 2001). Therefore, it is quite clear that, with the continued increase in the size of the aging population, AD remains and will be a major economic and social concern worldwide and a tremendous unmet medical need.

Modern DNA, RNA, and protein expression technologies are revolutionizing our view and understanding of current neurological diseases, such as AD, and enable researchers to analyze the concurrent expression patterns of very large numbers of genes. These new high-throughput genomic and proteomic technologies (commonly referred to as the “Systems Biology” approach; Hood et al., 2004), such as DNA and protein microarrays, allow for the simultaneous study of thousands of genes and protein end products, and their alterations in regulation and modulation patterns in relation to disease state, time, and tissue specificity. However, due to post-transcriptional and post-translational (e.g., phosphorylation and glycolysation of proteins) modifications, the relationship between the level of mRNA and those of the protein end product is not always the same. In many instances, there is a positive correlation between the mRNA and protein levels in a tissue sample, but often there is no correlation, and frequently a negative correlation is observed (Lewandowski and Small, 2005). Thus, protein expression profiling is necessary as a follow-up procedure to any DNA microarray finding.

Advances in molecular genetic studies in the past two decades have allowed the identification of several genetic loci associated with AD. AD-related genes have been classified into genes with demonstrated mutations following a Mendelian inheritance pattern (i.e., mutational genetics), such as amyloid precursor protein (APP), presenilin-1 (PS1), and presenilin-2 (PS2), susceptibility genes or polymorphic loci potentially contributing to AD predisposition (i.e., susceptibility genetics), such as apolipoprotein E (APOE), alpha-2-macroglobulin (A2M), low density lipoprotein-related protein-1 (LRP1), interleukin-1 (IL1), and angiotensin I converting enzyme (ACE), and defective genes linked to mitochondrial DNA (mtDNA) with heteroplasmic transmission (reviewed in Cacabelos, 2002).

Recently, a triple-transgenic mouse model of AD (3xTg-AD) harboring three mutant genes, namely, β-amyloid precursor protein (βAPP_{Swe}), presenilin-1 (PS1_{M146V}), and tau_{P301L}, has been developed that uniquely expresses both of the hallmark neuropathological lesions associated with AD, that is, the Aβ plaques and neurofibrillary tangles (NFT) (Oddo et al., 2003a,b). These mice develop the Aβ and tau pathologies with a temporal- and regional-specific profile that closely resembles their development in the human AD brain. In fact, it has been observed that the extracellular β-amyloid deposits initiate in the cerebral cortex and with aging progress to the hippocampus, whereas the tau pathology first appear in the hippocampus and progress to the cortex (Oddo et al., 2003a,b). This observed pattern of Aβ deposition developing prior to tau pathology is also consistent with the current widely accepted amyloid cascade hypothesis of AD. According to a recent study (Billings et al., 2005), at 2-months old, the prepathologic 3xTg-AD mice are cognitively normal. However, at 4-months, the mice manifest the earliest cognitive impairment as a deficit in long-term retention, which correlates with the accumulation of intraneuronal Aβ in the hippocampus and amygdala. The results from this study strongly suggest the intraneuronal accumulation of Aβ in the onset of cognitive dysfunction in the 3xTg-AD mice. On the other hand, the earliest sign of tau pathology in the 3xTg-AD mice appears with the accumulation of tau in the somatodendritic compartment at 6-months of age. The tangle pathology is quite advanced and apparent by 18 to 20-months with different silver stains and immunoreactive with several phosphor-specific tau antibodies, such as AT8 and PHF-1 (Oddo, et al., 2003b; Oddo, et al., 2005).

In addition, since the 3xTg-AD mice are generated from simultaneously microinjecting two transgenes (i.e., βAPP and tau) into single-cell embryos from homozygous PS1_{M146vV} knockin mice, rather than crossing independent lines, the mice all have the same genetic background. Compared to crossbreeding, this approach offers several major advantages. For example, deriving a large colony of the 3xTg-AD is straightforward, cost-effective, and does not require extensive genotyping of the progeny. Moreover, the easy propagation of this transgenic line facilitates their crossing to other transgenic or gene-targeted mice to assess the impact of other genotypes on the neuropathological or physiological phenotype. Finally, since multiple transgenes are introduced into an animal without altering or mixing the background genetic constitution, an important confounding variable is avoided. This provides crucial parameter control for behavioral, genomic, proteomic, and vaccine-based experiments.

### SUMMARY OF THE DISCLOSURE

According to a feature of the present disclosure, there is provided a business method including novel enhanced processes for the identification of biomarkers for Alzheimer's disease (AD), the method comprising an application of a systems biology approach to Alzheimer's disease to lead to immediate short-term development of novel clinical diagnostic tools, such as bioconjugated QD biosensors, for the sensitive and early detection of AD within the human population discovery and development of new therapeutic agents, using these types of tools. Determining whether candidate biomarker genes and candidate biomarker proteins are predictive of the progression of AD, and development of personalized medicines (i.e., pharmacogenomics) for individuals within the human population worldwide in need of such treatment, for example, those suffering from AD.

According to another feature of the present disclosure, there is provided an improved process for discovering, mining and otherwise addressing indicia selected from the group consisting of genes, proteins, metabolites and related biomarkers associated with neurodegenerative disease states comprising identifying aspects of disease to be investigated, such as signature biochemical networks, deposits of amyloid proteins, neurofibrillary tangles, and related expressions. Reviewing the same against statistical measures, optionally, or other bioinformatic tools, standards and systems, validating and investigating subject indicia for example by cross-referencing their presence in other bodily fluids, and providing indicators for responses of animals, similar to or modeling those in need of treatment to specified therapeutics, dosages and treatment regimens, such as bioconjugated quantum dot nanocrystals, linked to biological molecules and capable of stable fluorescent light emission and multiplexing.

According to yet another feature of the present disclosure, there is provided a nanosytems biological approach to development of novel clinical diagnostic tools for treating neurodegenerative disease which comprises utilizing a gene expression profiling protocol in conjunction with phenotypic analysis to understand subject gene expression patterns and neuroanatomical alterations, measuring protein expression levels to identify protein-protein interactions of identified candidates, performing immunohistochemical, gene and protein expression analyses in conjunction with monitoring progression and pathogenesis behaviorally, functionally analyzing expressed data to discover novel molecular networks in comparison to user-defined lists to known biologicval association networks databases, and introducing new molecular diagnostic assays for accurate, predictive and early and pre-symptomatic detection of neurodegenerative disease in the human population.

### DETAILED DESCRIPTION OF THE DISCLOSURE

The contents of Appendix A is hereby incorporated by reference as if fully disclosed herein.

The present disclosure includes methods for the identification of biomarkers associated with Alzheimer's disease (AD). Biomarkers identified according to the methods disclosed herein can be detected in diagnostic and prognostic assays, allowing AD to be diagnosed earlier and more accurately than was previously possible, and also providing a clinician with more prognostic information than current assays. In addition, biomarkers identified according to the methods disclosed herein can serve as drug targets for the identification of new therapeutic agents for the treatment of AD.

As used herein, the term “biomarker” includes a nucleic acid(s), protein(s), or metabolite(s) whose presence, absence, or, level of expression is a measure of the progression of AD or of the likelihood of developing AD. A biomarker may comprise a single nucleic acid, protein, or metabolite, or it may comprise a plurality of nucleic acids, proteins, and/or metabolites whose presence, absence, or levels of expression collectively provide a measure of the progression of AD or of the likelihood of developing AD.

The methods included in the present disclosure involve the phenotypic analysis of 3xTg-AD mice and control mice. In one aspect, the methods involve the use of phenotypic analysis techniques. The individual phenotypic analysis techniques include, but are not limited to: immunochemical analysis of proteins that are relevant to the pathogenesis and progression of AD; analysis of behaviors that are relevant to the pathogenesis and progression of AD; RNA expression (gene) analysis for the determination of candidate biomarker genes that are differentially expressed in 3xTg-AD mice in comparison to control mice; and protein expression (proteomic) analysis for the determination of candidate biomarker proteins that are differentially expressed in 3xTg-AD mice in comparison to control mice. Preferably at least one immunochemical assay, at least one behavioral assay, at least one RNA expression assay, and at least one protein expression assay is performed. The assay results are then subjected to bioinformatic analysis in order to identify biomarkers that provide a measure of the progression of AD or a measure of the likelihood of developing AD. Specifically, by correlating the protein expression analysis results and/or the RNA expression analysis results with the progression and pathogenesis of AD in 3xTg-AD (as determined using the immunochemical and behavioral assays), and further through comparison with control mice, biomarkers for AD are identified.

In one embodiment, the plurality of phenotypic analysis techniques are performed at a plurality of different time points in the life of 3xTg-AD mice, and the results are compared with the results obtained from performing the same plurality of phenotypic analyses on age, gender, and genetic background-matched non-transgenic control mice. For example, the plurality of phenotypic analyses may be performed at 2, 4, 6, 8, 10, 12, 15, and 18 months of age in both 3xTg-AD mice and control mice.

For each time point, a plurality of 3xTg-AD mice and a plurality of control mice may be used. For example, three mice 3xTg-AD mice and three control mice may be used at each time point.

In one embodiment, behavioral analysis of the 3xTg-AD mice is performed at each time point using learning retention and memory paradigms. Examples of such paradigms include the Spontaneous Alternation Y Maze Task (Holcomb et al., 1998) and Spatial Reference Water Maze Training. In this way, it is possible to monitor AD pathology and progression from a behavioral standpoint at each time point. Following the behavioral analysis, the 3xTg-AD and control mice can be prepared for immunochemical analysis, RNA expression analysis, and protein expression analysis.

In one embodiment, immunochemical analysis is performed by detecting pathologically-relevant proteins at one or more of the time points in 3xTg-AD and control mice. In this way, it is possible to monitor AD pathology and progression from a biochemical standpoint at each time point. Immunochemical analysis includes assays for the expression level of individual proteins in the brain, such as Enzyme-Linked Immunosorbent Assays (ELISA), and also includes immunohistochemical assays in which the localization of individual proteins to specific tissues and cells, and the expression levels of those proteins in those tissues and cells, is determined using fixed brain tissue sections. For example, immunohistochemical assays may be performed by immunolabeling using a primary antibody and a fluorescent secondary antibody, followed by image analysis using a microscope equipped with fluorescence optics, such as a laser scanning confocal microscope.

In one embodiment, ELISA assays are performed using antibodies specific for Aβ₁₋₄₀ (for example, using the BNT77/BA27 antibody system known in the art) and Aβ₁₋₄₂ (using the BNT77/BC05 antibody system known in the art). In one embodiment, immunohistochemical assays are performed using the following antibodies: anti-Aβ 6E10 and 4G8 (Signet Laboratories, Dedham, Mass.), anti-Aβ 1560 (Chemicon), A11 (Kayed et al., 2003), anti-APP 22C11 (Chemicon), anti-Tau HT7, AT8, AT180 (Innogenetics), Tau C17 (Santa Cruz), Tau 5 (Calbiochem), anti-GFAP (Dako), and anti-actin (Sigma).

Preferably, a multifactor ANOVA (Analysis of Variance) algorithm is used to analyze behavior scores of the mice and/or to analyze immunohistochemistry scores. Post hoc Fisher's Protected Least Significant Difference (PLSD) tests may be performed to determine significance of differences between the groups when appropriate.

In one embodiment, RNA expression analysis is performed after the behavioral analysis on mRNA extracted from the brains, or from sub-regions of the brain, of 3xTg-AD and control mice at one or more time points. For example, the mRNA may be extracted from pre-dissected hippocampal (particularly the dentate gyrus (DG) subregion) and frontal-cortical regions. The DG is the hippocampal subregion that has been found to be most sensitive to advancing age in several species, including rodents (Small et al., 2004). The identity and expression level of the extracted mRNA may be determined using any RNA analysis technique known in the art. For example, mRNA may be analyzed using whole genome or sub-genomic expression microarrays, such as the Affymetrix 430 2.0 (Santa Clara, Calif.) mouse whole genome expression microarray. Cerebellar subregions may also be analyzed as negative controls. Quantitative Reverse transcription polymerase chain reaction (RT-PCR) analysis using gene specific primers, including real time quantitative RT-PCR, may then be used to confirm the microarray findings. Alternatively, RT-PCR may be used independently to identify expressed RNA and to quantitate RNA expression. Using the aforementioned RNA analysis techniques, candidate gene biomarkers are chosen based on significantly differing RNA expression levels in 3xTg-AD mice in comparison to control mice samples at any of the time points.

In one embodiment, laser capture microdissection (LCM) is used to obtain homogenous populations of cell from heterogeneous brain tissue samples for use in RNA expression analysis. For example, fresh-frozen areas of the hippocampus and frontal section can be sectioned, fixed in acetone, and Nissl stained for neuronal identification based on neuronal morphology and cytoarchitecture. Once a region of interest is selected in the stained section, a laser capture microscope, for example a Arcturus PixCell I (Arcturus Engineering, Mountain View, Calif.) is used to capture particular neuron populations. The dissected tissue is then transferred to a plastic membrane and recovered in a microcentrifuge tube for subsequent nucleic acid extraction and microarray analysis and/or RT-PCR analysis, as described above.

In one embodiment, protein expression analysis is performed using two-dimensional (2-D) gel electrophoresis of brain cell lysates, such as cell lysates from hippocampal and frontal cortical samples, of 3xTg-AD and control mice. For example, in the first dimension, isoelectric focusing (IEF) is employed to separate proteins based on their intrinsic charge characteristics, and in the second dimension, based on protein mass via sodium dodecyl sulfate-polyacrylamide gel electrophoresis (SDS-PAGE). Candidate protein biomarkers are initially chosen based on significantly differing protein expression levels in 3xTg-AD mice in comparison to control mice samples at any of the time points. Protein levels on 2-D gels may be determined, for example, using densitometry techniques known in the art.

In one embodiment, protein spots resolved by 2-D gel electrophoresis (including those that comprise candidate protein biomarkers as initially determined by, for example, gel densitometry) are analyzed using mass spectrometric methods known in the art. Typical mass spectrometric (MS) methods for protein identification involve the recovery of peptides derived by in-gel digestion (using trypsin) of protein spots excised from the 2-D gels. The recovered peptides can be analyzed by matrix-assisted laser desorption/ionization time-of-flight (MALDITOF) to generate peptide maps. Alternatively MALDI-TOF-TOF or liquid-chromatography electrospray ionization (LC-ESI-MS-MS) on a quadrupole time-of-flight (QTOF) type instrument can be used to obtain sequence information on individual peptides. Both the mapping and sequence information is subjected to appropriate database searching to identify the candidate protein biomarkers from which the peptides were derived. In addition, Fourier transform ion cyclotron (FT-MS) may also be used, for example, for “top-down” sequencing that may obviate the need for the digestion step.

In one embodiment, the identified candidate protein biomarkers are further analyzed for functional parameters. For example, the candidate protein biomarkers may be analyzed for protein-protein interactions, using protein microarrays, including mouse protein microarrays and human protein microarrays. Briefly, the candidate protein biomarkers are contacted with a microarray comprising a plurality of known proteins arrayed at specific locations on a solid support. Functional protein microarrays can be used to reproduce most major types of interactions and enzymatic activities seen in biochemical pathways. (Predki et al., 2004) Interaction between a candidate biomarker and one or more of the proteins on the microarray can then be detected by detecting binding of the candidate protein biomarker to one or more of the locations on the microarray, which in turn reveals the interactions that the candidate protein biomarker has in vivo. Commercial protein microarray systems, such as the ProtoArray system (Invitrogen, Carlsbad, Calif.), may be advantageously employed in such embodiments.

By comparing the expression of the candidate gene biomarkers and the candidate protein biomarkers with the measures of AD progression determined by the behavioral assays and the immunochemical assays, it is possible to identify biomarkers for AD progression.

In another aspect of the disclosure, the biomarkers identified according to the methods disclosed herein can be analyzed using bioinformatics databases in order discover novel molecular networks involved in AD.

Given that transgenic mouse models of AD can be scientifically controlled with great precision in comparison with human research efforts, biomarker discovery in the 3xTg-AD mice according to the methods described herein can progress very quickly. Such biomarker discovery endeavors via a transgenic mouse model of the disease will greatly facilitate the process of drug discovery and development and early detection of AD in humans. For example, biomarkers of AD elucidated through the methods of the disclosure can be immediately validated by cross-comparison with peer-reviewed, published human data, and subsequently by directly assaying representative human brain tissue.

The following examples are intended only to illustrate the methods of the present disclosure and should in no way be construed as limiting the subject disclosure.

### EXAMPLES

### Example 1

### Mice and Surgical Procedures

Similar to the methodology described in Oddo et al. (2003a,b), 3xTg-AD harboring APP_{Swe}, PS1_{M146V}, and tau_{P301L} transgenes, which were generated by simultaneous microinjection of two independent transgene constructs encoding human APP_{Swe} (i.e., Swedish familial mutation) and tau_{P301L} into the pronuclei of single-cell embryos harvested from mutant homozygous PS1_{M146V} knockin mice. The PS1 knockin mice were originally generated on a hybrid 129/C57BL/6 background (Guo et al., 1999). Southern blot analysis of tail DNA is subsequently used to identify transgenic mice (LaFerla et al., 1995; Sugarman et al., 2002).

### Example 2

### Behavioral Measures

Spontaneous Alternation Y Maze Task. As previously described (Holcomb et al., 1998), this learning paradigm involves hippocampal circuits that direct spatial working memory and bypasses the need for any training, reward, or punishment. The Y maze apparatus is comprised of three acrylic arms at 120° angles to one another. The dimensions of each arm are as follows: 40 cm length, 17 cm height, 4 cm width at the bottom and 13 cm width at the top. Each mouse, being placed in the center of the maze, is given 8 minutes to navigate through the maze freely. The sequence of entry and number of maze arms entered (entry defined as having all hind paws within the arm) is recorded. Percentage alternation is calculated as described in the above-mentioned literature.

Spatial Reference Morris Water Maze (MWM) Training. Mice are trained to swim to a submerged (and functionally invisible) 14 cm diameter circular clear Plexiglas platform. After being released from one randomly selected start point (of 4 designated start points), mice are allowed 60 sec to locate and escape onto the platform, after which they are manually guided to the platform on which they remain for 10 sec. During the inter-trial interval, the mice are placed under a warming lamp in a holding cage for 25 sec. All mice are trained to criterion (<20 sec mean escape latency) to control for memory differences due to lack of task learning. Cued platform training is utilized to control for visual ability and intact striatal mediated learning (Billings et al., 2005). This consists of four consecutive trials daily in which starting position (along edge of tank) and platform location is altered for each trial. Retention of the spatial reference training is measured at 1.5 hr and at 24 hr post last training trial, consisting of a 60 sec free swim without platform (following Billings et al., 2005). The parameters assessed during the retention trials include initial latency to cross the platform location, number of platform location crosses, and time spent in quadrant opposite platform.

### Example 3

### ELISA Quantitation of Brain Aβ Levels

After behavioral tests are completed as in Example 2, the Aβ₁₋₄₀ and Aβ₁₋₄₂ levels are measured using a sensitive sandwich enzyme-linked immunosorbent assay (ELISA) system (Duff et al., 1996; Miller et al., 2003). Frozen hemibrains are extracted in 0.2% diethlyamine with 50nM NaCl and centrifuged at 20,000×g for 1 hr at 4° C. to remove insoluble material. The resulting supernatant fractions are analyzed using the well-known BNT77/BA27 and BNT77/BC05 antibody systems to detect Aβ₁₋₄₀ and Aβ₁₋₄₂, respectively. These sandwich ELISAs are known to recognize both human and mouse Aβ₁₋₄₀ and Aβ₁₋₄₂ with equivalent sensitivities.

### Example 4

### Immunohistochemistry

Initial processing follows the protocol as previously described (Oddo et al., 2003a,b; Billings et al., 2005). Briefly, mice are sacrificed by CO₂ asphyxiation, and the brains are rapidly removed and fixed for 48 hr in 4% paraformaldehyde. Free-floating (5 μm thick) sections are mounted onto silane-coated slides and processed with the following antibodies: anti-Aβ 6E10 and 4G8 (Signet Laboratories, Dedham, Mass.), anti-Aβ 1560 (Chemicon), A11 (Kayed et al., 2003), anti-APP 22C11 (Chemicon), anti-Tau HT7, AT8, AT180 (Innogenetics), Tau C17 (Santa Cruz), Tau 5 (Calbiochem), anti-GFAP (Dako), and anti-actin (Sigma). Primary antibodies are applied at dilutions of 1:3000 for GFAP; 1:1000 for 6E10; 1:500 for 1560, AT8, AT180, and Tau 5; and 1:200 for HT7. Sections are then developed with diaminobenzidine (DAB) substrate using the avidin-biotin horseradish peroxidase system (Vector Labs).

To achieve highly resolved target-specific images, a two-way fluorescent immunolabeling technique, involving application of a primary antibody followed by a fluorescent secondary antibody, is implemented. After initial immunohistochemical processing, tissue is incubated for 1 hr in fluorescently labeled anti-mouse 20 antibody (Alexa 488; 1:200; Molecular Probes Inc., Eugene, Oreg.). Slices are then incubated for 20 min in TOTO-13 iodide to add nuclear markers (Molecular Probes Inc.; 1:200 in PBS). Confocal images are subsequently be captured on an MRC 1024 (BioRad, Hercules, Calif.) confocal system.

### Example 5

### Laser Capture Microdissection (LCM) of Tissue Sections

To obtain homogenous populations of cells from heterogeneous hippocampal and frontal cortical tissue sections, the method of laser capture microdissection (LCM) is employed. Selected fresh-frozen areas of the hippocampus and frontal cortex are sectioned at 6 μm thick, briefly fixed with acetone, and Nissl stained for neuronal identification based on neuronal morphology and cytoarchitecture. Once a region of interest is selected in the stained section, an Arcturus PixCell I laser capture microscope (Arcturus Engineering, Mountain View, Calif.) with a beam size of 30 μm, which is sufficient to capture cell clusters containing as few as 20 cells, is used to capture particular neuron populations. In addition, a Leica LS-AMD (Leica Microsystems Bannockburn, Ill.) outfitted with fluorescent optics and a minimum beam width of less than 1 μm is used for selective visualization and capture of stained neurons. The dissected tissue is then transferred to a plastic membrane (“cap”; Arcturus Engineering) and recovered in a microcentrifuge tube for subsequent nucleic acid extraction and microarray analysis. Tissue contaminants are removed from the transfer caps with Arcturus's CapSure sticky pads. All procedures are performed under RNAse free conditions.

### Example 6

### RNA Isolation

Total RNA from representative hippocampal and frontal cortical areas of mice from each of the four sub-groups is extracted using the TRIzol reagent according to the manufacturer's specifications (Invitrogen, Carlsbad, Calif.). Samples are first homogenized in TRIzol reagent for 30 sec. After mixing with chloroform, the samples are then centrifuged for 15 min at 12,000×g at 4° C. Isopropanol is subsequently be added to the aqueous phase for RNA precipitation. This precipitation mix is centrifuged for 10 min at 12,000×g at 4° C. The RNA pellet is then washed once with cold 75% ethanol and air dried for 10 min. Finally, total RNA is resuspended in RNAse-free water. Total RNA is subsequently resuspended in nuclease-free water and subjected to TURBO DNase treatment (Ambion, Austin, Tex.). Genomic DNA-free RNA is further purified by an RNeasy column (Qiagen, Valencia, Calif.). Finally, RNA is eluted from the column using nuclease-free water.

### Example 7

### DNA Microarray Hybridization and Analysis

The Affymetrix Mouse Genome 430 2.0 Arrays (Affymetrix, Santa Clara, Calif.) that contain 45,101 probe sets are used to examine gene expression patterns. Following isolation of total RNA (mRNA) from hippocampal and frontal cortical brain tissue from each animal, all subsequent technical procedures, including quality control of RNA, labeling with biotin-rNTPs, hybridization, and scanning of the arrays are performed according to methods known in the art.

Expression level of genes is normalized by GCRMA (Wu and Irizarry, 2004) followed by identification of statistically significant genes by Cyber-T/ANOVA (http://visitor.ics.uci.edu/genex/cybert/). The GeneSpring software (Agilent Technologies) is then used to cluster and visualize changes of gene expression patterns in correlation with genotypes and ages of mice.

### Example 8

### Quantitative Real-Time RT-PCR

Quantitative real-time reverse transcription-polymerase chain reaction (RT-PCR) is performed to confirm the microarray findings as described elsewhere (Saura et al., 2004). Briefly, part of the RNA samples used for the microarray studies is treated with DNase I and reverse transcribed in the presence of random hexamers. PCR reactions are performed using SYBR Green PCR Master Mix in an ABI PRISM 7700 Sequence Detector (Applied Biosystems) with 10 μl of diluted (1:25) cDNA and gene-specific primers. Reactions are performed in duplicate and the threshold cycle values normalized to 18 S RNA. Electrophoresis is then used to confirm the correct sizes of the PCR products. Alternatively, a melting curve of each PCR reaction is generated to verify a single specific PCR product.

### Example 9

### Two-Dimensional (2-D) Gel Electrophoresis and Mass Spectrometry (MS)

The Zoom IPGRunner (Invitrogen, Carlsbad, Calif.) system is used for two-dimensional (2D) gel electrophoretic protein separation, as described previously (Gorg et al., 2005). Briefly, proteins are separated in a two-step process. In the first dimension, isoelectric focusing (IEF) is employed to separate proteins based on their intrinsic charge characteristics, and in the second dimension, based on protein mass via sodium dodecyl sulfate-polyacrylamide gel electrophoresis (SDS-PAGE).

Typical mass spectrometric (MS) methods for protein identification involve the recovery of peptides derived by in-gel digestion (using trypsin) of protein spots excised from 2-D gels. The recovered peptides can be analyzed by matrix-assisted laser desorption/ionization time-of-flight (MALDITOF) to generate peptide maps. Alternatively MALDI-TOF-TOF or liquid-chromatography electrospray ionization (LC-ESI-MS-MS) on a quadrupole time-of-flight (QTOF) type instrument can be used to obtain sequence information on individual peptides. Both the mapping and sequence information is subjected to appropriate database searching to obtain the identifications of the proteins. In addition, Fourier transform ion cyclotron (FT-MS) may also be used, for example, for “top-down” sequencing that may obviate the need for the digestion step. Initial quantification, to look for up and down regulated proteins, can by accomplished on the 2-D gels using densitometry.

### Example 10

### Protein Arrays

In order to characterize specific protein interactions of differentially expressed proteins, human ProtoArray protein microarrays (Invitrogen, Carlsbad, Calif.) are employed to screen labeled probes against over 5,000 unique proteins. Functional protein microarrays present an important new tool ideally suited to the mapping of biological pathways. Protein microarrays were developed to provide miniaturized high-throughput tools to study protein function, expression, and post-translational modifications. Functional protein microarrays can be used to reproduce most major types of interactions and enzymatic activities seen in biochemical pathways. Because of the unique ability to address different aspects of biological pathways, functional protein microarray technology is primed to make significant contributions to the understanding of disease pathways for both basic and drug research (Predki et al., 2004). All proteins are expressed in a baculovirus system to maintain post-translational modifications, and purified under native conditions to preserve maximum functionality and protein structure. Briefly, human protein microarrays containing over 5,000 full-length proteins are screened with a probe containing single V5 or biotin tags. Interacting proteins are then detected using AlexaFluor labeled anti-V5 antibody or Alexa Fluor labeled streptavidin. Data analysis can be performed using a manual analysis of the arrays to identify significant signals on the slide. Alternatively, a software program, such as the ProtoArray Prospector (Invitrogen, Carlsbad, Calif.), is used for analysis of protein-protein interaction data on Invitrogen Protoarrays. This is a freeware tool that quickly identifies statistically significant signals on the arrays. To date, approximately 80% of interactions that are detected by a solution-based assay (gel mobility shift) are also observed on the ProtoArrays.

In addition, mouse candidate biomarkers are run against human protein microarrays since the 3xTg-AD mice contain human transgene variants (Oddo et al., 2003a,b), and alternative splicing is highly conserved and exon sequence homology among mice and humans is quite strong (Sugnet et al., 2004; Thanaraj et al., 2003). Thus, candidate molecules, such as proteins identified by 2-D gel and MS screening of 3xTg-AD vs. control brain tissue, or molecules from other sources, are hybridized to human ProtoArray protein microarrays to attempt to reveal novel protein-molecule interactions. This information is used to further map biochemical pathways in AD pathogenesis, potentially uncovering additional novel AD biomarkers.

### Example 11

### iTRAQ Analysis

To determine the relative quantitative protein expression profiles of brain tissue samples from the hippocampal and frontal cortical areas of 3xTg-AD mice as compared to controls, the techniques of isobaric Tagging for Relative and Absolute protein Quantification (iTRAQ, Applied Biosystems, Foster City, Calif.) coupled with Multidimensional Protein Identification Technology (MudPIT) will be performed (DeSouza et al., 2005).

Protein extracts (approximately 150 mg) from up to 4 different experimental and control groups (2 shown on chart from FIG. 5) are reduced, alkylated, and digested with trypsin in an amine-free buffer system, in parallel (Ross et al., 2004). The resulting peptides are then labeled with the iTRAQ Reagents for 1 hr at room temperature. Upon completion of labeling, the samples are combined and directly analyzed by 2-dimensional High Performance Liquid Chromatography (2D HPLC), including separation by strong cation exchange (SCX) coupled with fused silica capillaries and reverse phase chromatography (RP) for optimal peptide separation. An LC Packings UltiMate LC system (Dionex) will be used to analyze array-plated samples that is interfaced offline onto a 4700 Proteomics Analyzer (Sciex/Appplied Biosystems) (Ross et al., 2004). Spectra from the 4700 Proteomics Analyzer will be loaded into the GPS Explorer software (Applied Biosystems) and searched against a murine protein database with trypsin specificity using the MASCOT search engine (www.matrixscience.com) (Zhang et al., 2005). Data will be normalized to the vehicle-treated control values for comparison with experimental groups. To allow for the identification of potentially unlabelled peptides, protein database searches will also be performed with the iTRAQ Reagent derivatives as variable modifications. Finally, a paired, two-tailed Student's t-test will be performed for statistical analysis of the data.

### Example 12

### Statistical Analysis

A multifactor ANOVA, including genotype and age, is used to analyze behavior scores of the mice. Post hoc Fisher's PLSD tests are performed to determine significance of differences between the groups when appropriate. In addition, the immunohistochemistry scores are analyzed by ANOVA with results being considered significant when p<0.05.

For microarray data analysis, Cyber-T/ANOVA software is used to perform a regularized F-test with a Bayesian statistical framework. A beta-mixture modeling method is performed to determine posterior probability of differential expression (PPDE) for each gene at all pair-wise comparisons. This analysis allows identification of differentially expressed genes among different age and genotype groups with high levels of confidence. Post hoc Tukey test is performed for all pair-wise comparisons between genotypes and ages to identify differentially expressed genes.

### Example 13

### Discovery of Novel Molecular Networks

Differentially expressed genes identified by Cyber-T/ANOVA software are further analyzed by MetaCore (GeneGo, St Joseph, Mich.). MetaCore is a platform that has the largest systems biology proprietary manually curated database with a suite of software tools for analysis. GeneGo uses Ph.D. level annotators that are employees to read full text articles to populate the database with genes, proteins, hormones, compounds, metabolites and transcriptional factors, mechanisms of interaction, direction, and links to papers. MetaCore has the unique ability to provide merged metabolic and signaling pathway networks as well as a metabolic parser for visualizing MS concentration data in the context of canonical maps and pathways. Furthermore, MetaCore has the ability to concurrently visualize gene expression and proteomics data as well as multiple time points, dosages, and treatments to identify key functions and pathways that distinguish biological states. Deeper analysis can be completed by working with tissue, subcellular localization, interaction, ortholog, and functional process filters as well as understanding other drug targets in the networks. MetaCore can also build disease specific signature networks as a starting point for investigation.

### REFERENCES

The following references are cited within the disclosure. Each reference is specifically incorporated herein by reference in its entirety.

Alzheimer's Association. 2001. Medicare and Medicaid costs for people with Alzheimer's disease. Washington, D.C.: The Lewin Group. p. 1.

Auld, D. S., Kornecook, T. J., Bastianetto, S., and Quirion, R. 2002. Alzheimer's disease and the basal forebrain cholinergic system: relations to beta-amyloid peptides, cognition, and treatment strategies. Progress in Neurobiology. 68:209-245.

Billings, L. M., Oddo, S., Green, K. N., McGaugh, J. L., and LaFerla, F. M. 2005. Intraneuronal Aâ causes the onset of early Alzheimer's disease-related cognitive deficits in transgenic mice. Neuron. 45:675-688.

Cacabelos, R. 2002. Pharmacogenomics in Alzheimer's disease. Mini Reviews in Medicinal Chemistry. 2(1):59-84. Crentsil, V. 2004. The pharmacogenomics of Alzheimer's disease. Ageing Research Reviews. 3(2):153-169.

Duff, K., Eckman, C., Zehr, C., Yu, X., Prada, C. M., Perez-tur, J., Hutton, M., Buee, L., Harigaya, Y., Yager, D., et al. 1996. Increased amyloid-beta42(43) in brains of mice expressing mutant presenilin 1. Nature. 383:710-713.

Ernst, R. L. and Hay, J. W. 1994. The U.S. economic and social costs of Alzheimer's disease revisited. American Journal of Public Health. 84(8):1261-1264.

Gorg, A., Weiss, W., and Dunn, M. J. 2005. Current two-dimensional electrophoresis technology for proteomics. Proteomics. 5(3):826-827.

Guo, Q., Fu, W., Sopher, B. L., Miller, M. W., Ware, C. B., Martin, G. M., and Mattson, M. P. 1999. Increased vulnerability of hippocampal neurons to excitotoxic necrosis in presenilin-1 mutant knock-in mice. Nature Medicine. 5:101-106.

Hardy, J. and Selkoe, D. J. 2002. The amyloid hypothesis of Alzheimer's disease: progress and problems on the road to therapeutics. Science. 297:253-256.

Hock, B. J. and Lamb, B. T. 2001. Transgenic mouse models of Alzheimer's disease. Trends in Genetics. 17(10):S7-S12.

Holcomb, L., Gordon, M. N., McGowan, E., Yu, X., Benkovic, S., Jantzen, P., Wright, K., Saad, I., Mueller, R., Morgan, D., et al. 1998. Accelerated Alzheimer-type phenotype in transgenic mice carrying both mutant amyloid precursor protein and presenilin 1 transgenes. Nature Medicine. 4:97-100.

Hood, L., Heath, J. R., Phelps, M. E., and Lin, B. 2004. Systems biology and new technologies enable predictive and preventative medicine. Science. 306(5696):640-643.

Kayed, R., Head, E., Thompson, J. L., McIntire, T. M., Milton, S. C., Cotman, C. W., and Glabe, C. G. 2003. Common structure of soluble amyloid oligomers implies common mechanism of pathogenesis. Science. 300:486-489. Koppel, R. 2002. Alzheimer's Disease: The costs to U.S. businesses in 2002. Washington, D.C.: Alzheimer's Association.

LaFerla, F. M., Tinkle, B. T., Bieberich, C. J., Haudenschild, C. C., and Jay, G. 1995. The Alzheimer's A beta peptide induces neurodegeneration and apoptotic cell death in transgenic mice. Nature Genetics. 9:21-30.

Lewandowski, N. M. and Small, S. A. 2005. Brain Microarray: Finding Needles in Molecular Haystacks. Journal of Neuroscience. 25(45):10341-10346. 10

Miller, B. C., Eckman, E. A., Sambamurti, K., Dobbs, N., Chow, K. M., Eckman, C. B., Hersh, L. B., and Thiele, D. L. 2003. Amyloid-â-peptide levels in brain are inversely correlated with insulysin activity levels in vivo. Proceedings of the National Academy of Sciences, USA. 100:6221-6226.

Oddo, S., Caccamo, A., Kitazawa, M., Tseng, B. P., and LaFerla, F. M. 2003a. Amyloid depositon precedes tangle formation in a triple transgenic model of Alzheimer's disease. Neurobiology of Aging. 24:1063-1070.

Oddo, S., Caccamo, A., Shepherd, J. D., Murphy, M. P., Golde, T. E., Kayed, R., Metherate, R., Mattson, M. P., Akbari, Y., and LaFerla, F. M. 2003b. Triple-transgenic model of Alzheimer's disease with plaques and tangles: intracellular A\hat{a} and synaptic dysfunction. Neuron. 39:409-421.

Oddo, S., Caccamo, A., Tran, L., Lambert, M. P., Glabe, C. G., Klein, W. L., and LaFerla, F. M. 2005. Temporal profile of Ab oligomerization in an in vivo model of Alzheimer's disease: A link between Ab and tau pathology. Journal of Biological Chemistry. 10:1-14.

Predki, P. F. 2004. Functional protein microarrays: ripe for discovery. Current Opinion in Chemical Biology. 8(1):8-13.

Rice, D. P., Fox, P. J., Max, W., Webber, P. A., Lindeman, D. A., Hauck, W. W., and Segura, E. 1993. The economic burden of Alzheimer's disease. Health Affairs. 12(2):164-176.

Saura, C. A., Choi, S. Y., Beglopoulos, V., Malkani, S., Zhang, D., Shankaranarayana Rao, B. S., Chattarji, S., Kelleher, R. J., Kandel, E. R., Duff. K., Kirkwood, A., and Shen, J. 2004. Loss of presenilin function causes impairments of memory and synaptic plasticity followed by age-dependent neurodegeneration. Neuron. 42:23-36.

Selkoe, D. J. and Schenk, D. 2003. Alzheimer's disease: molecular understanding predicts amyloid-based therapeutics. Annual Review of Pharmacology and Toxicology. 43:545-584.

Small, S. A., Chawla, M. K., Buonocore, M., Rapp, P. R., and Barnes, C. A. 2004. Imaging correlates of brain function in monkeys and rats isolates a hippocampal subregion differentially vulnerable to aging. Proceedings of the National Academy of Sciences, USA. 101(18):7181-7186.

Sugarman, M. C., Yamasaki, T. R., Oddo, S., Echegoyen, J. C., Murphy, M. P., Golde, T. E., Jannatipour, M., Leissring, M. A., and LaFerla, F. M. 2002. Inclusion body myositis-like phenotype induced by transgenic overexpression of beta APP in skeletal muscle. Proceedings of the National Academy of Sciences, USA. 99:6334-6339.

Sugnet, C. W., Kent, W. J., Ares, M. Jr., and Haussler, D. 2004. Transcriptome and genome conservation of alternative splicing events in humans and mice. Pacific Symposium on Biocomputing 2004. 66-77.

Thanaraj, T. A., Clark, F., and Muilu, J. 2003. Conservation of human alternative splice events in mouse. Nucleic Acids Research. 31(10):2544-2552.

Wu, Z. and Irizarry, R. A. 2004. Stochastic Models Inspired by Hybridization Theory for Short Oligonucleotide Arrays. Proceedings of the 8th International Conference on Computational Molecular Biology. RECOMB. 11

### Appendix A

### Application of the Sytems Biology Approach to a Triple-Transgenic Mouse Model of Alzheimer's Disease for the Identification of Novel Diseases Biomakers

### By

### Shawn Mojtahedian, Ph.D. Innovative NeuroTechnologies, Inc. Irvine, Calif.

Specific Aims

Phase I (Preliminary Data to be Generated Roughly by the end of the Initial 6 Month Period)

Primary objectives: Application of the Systems Biology approach, including behavioral, phenotypic, genomic, proteomic, and bioinformatic analysis, of a triple-transgenic mouse model (3xTg-AD) of Alzheimer's disease (AD) to identify novel biomarkers (i.e., genes, protein, metabolites) associated with the disease. More specifically, gene and protein expression profiles from hippocampal and frontal cortical brain tissue from diseased 3xTg-AD mice will be compared to matched controls at various time points [i.e., 2 (pre-pathological), 6 (initial pathology), 15 (tangle pathology), and 24 (full pathology) months of age] and subsequently analyzed with various bioinformatic software (e.g., GeneSpring, ProtoArray Prospector, MetaCore) to delineate novel signature biochemical pathways and networks associated with AD. These preliminary sets of biomarkers, identified approximately by the end of the initial 6 month period (Phase I), will thus serve as the basis for the initial development of a novel highly sensitive, predictive, and reliable early detection assay (short-term, during the 2^{nd} and 3^{rd} year periods) and therapeutic agents (long-term, roughly 5-8 years) for AD, which will further be developed in Phase II of the project.

Phase II (To be Completed by the end of the 3^{rd} Year)

Primary objectives: Novel biomarkers for AD, such as new sets of proteins, identified in Phase I of the current project will be further validated and qualified as a clinical endpoint for AD in Phase II. To further validate and translate the data generated from the analysis of the 3xTg-AD mice brains to humans, a battery of assays (e.g., genomic and proteomic) for the panel of biomarkers identified from the signature biochemical networks will be paired with statistical measures to determine which, if any, of the identified panel of biomarkers are actually predictive of development of AD, and whether there is a specific combination of biomarkers within the panel that maximizes disease prediction sensitivity and specificity. We then will focus our efforts on implementing the newly discovered sets of biomarkers from the 3xTg-AD mice to develop a novel molecular diagnostic assay for the early detection of AD in the human population. For example, nucleic acid and/or protein biomarkers identified from the 3xTg-AD mouse brain will be further validated and investigated for their presence in readily accessible bodily fluids, such as blood, urine, tears, saliva, and/or cerebrospinal fluid (CSF), from individuals at high risk and susceptible of developing AD, and thereby will serve as an early detection and prognosis of the disease in these individuals. These predictive markers or biomarkers can also serve as indicators for responses of AD patients to specific drug treatments and for establishing the optimal drug or therapeutic dosage (i.e., individualized dosing).

One potential molecular diagnostic approach is to employ bioconjugated quantum dot (QD) nanocrystals or probes, which are linked to biological molecules like antibodies, peptides, proteins, or nucleic acids and contain bright and stable fluorescent light emission and multiplexing potential (i.e., capability to detect multiple disease markers simultaneously), against the newly identified sets of biomarkers (e.g., nucleic acids and proteins) from the 3xTg-AD mice brains to screen for the corresponding AD specific biomarkers in human blood and/or CSF samples. The current gold standards for detecting low copy-number nucleic acids and proteins in bodily fluids are PCR combined with a variety of molecular fluorophore assays and enzyme-linked immunosorbent assay (ELISA), respectively. However, the clinical use of these assays are labor intensive, time consuming, prohibitive of multiplexing, and expensive. QD biosensors offer a much higher level of sensitivity, spatial resolution, and multiplexing potential for detection of low-copy number nucleic acids and proteins in bodily fluids and tissue (Smith et al., 2006a, b).

These bioconjugated QD probes can also be used as high-resolution contrast markers for medical imaging tools, such as functional magnetic resonance imaging (fMRI) and positron emission tomography (PET), to noninvasively detect for the early presence of AD specific biomarkers within brain regions in vivo, such as the hippocampus and frontal cortex, of individuals at risk and susceptible to developing AD. Thus, the future use of bioconjugated QD probes as clinical diagnostic tools is invaluable for the early detection and classification of AD.

Commercialization Plan

Application of the Systems Biology approach to the 3xTg-AD mice will ultimately lead to the more immediate short-term development (within roughly 3 years) of novel clinical diagnostic tools, such as the abovementioned bioconjugated QD biosensors, for the sensitive, early, and pre-symptomatic detection of AD within the human population and the eventual long-term (approximately 5-8 years) discovery and development of new therapeutic agents. These efforts will in turn lead to the development of personalized medicines (i.e., pharmacogenomics) for individuals within the human population worldwide suffering from AD.

Abstract

Early events in the amyloid cascade model of Alzheimer's disease (AD) neuropathogenesis are presently a leading area of investigation of the disorder. A major issue with the known double-transgenic mouse models and other current animal models of AD is the incomplete expression of classic AD phenotypes relative to human development of the disease. This issue has been addressed with the introduction of a triple-transgenic mouse model of AD (3xTg-AD). This model, which harbors PS1_{M146V}, APP_{Swe}, and tau_{P301L} homozygous transgenes is, to the extent of our knowledge, the only current animal model that exhibits both of the hallmark neuropathological alterations related with AD, namely, extracellular β-amyloid (Aβ) plaques and intracellular neurofibrillary tangles (NFTs). In the present study, we will apply a comprehensive Systems Biology approach to the 3xTg-AD model to identify novel and specific biomarkers associated with AD for potential early diagnosis and therapeutic agents. We will use a broad and deep gene expression profiling protocol, mainly through the use of Affymetrix DNA microarrays, in conjunction with phenotypic (i.e., immunohistochemical and behavioral) analyses to further our understanding of the gene expression patterns and neuroanatomical alterations underlying AD. In addition, protein expression levels will be measured primarily with the use of the iTRAQ reagent method coupled with Multidimensional Protein Identification Technology (MudPIT) as a follow-up to the DNA microarray findings, and functional protein microarrays (i.e., Invitrogen's ProtoArrays) will be employed to examine further for protein-protein interactions of identified candidate proteins. Representative tissue samples taken from frontal cortical and hippocampal areas, particularly the entorhinal cortex (EC), of each mouse brain will be used for immunohistochemical, gene, and protein expression analyses. We will examine the 3xTg-AD mice against control groups at specific time points [i.e., 2 (pre-pathological), 6 (initial pathology), 15 (tangle pathology), and 24 (full pathology) months of age] to monitor the progression and pathogenesis of the disease, further implementing behavioral measures to assess memory and learning retention alterations. A multifactor ANOVA, including genotype and age, will be used to analyze the behavior scores of all mice, and the Cyber-T/ANOVA and Agilent's GeneSpring GX programs will be employed for statistical analysis of the gene expression data. GeneGo's MetaCore integrated software suite will also be used for a functional analysis of the gene and protein expression data for the discovery of novel molecular networks by comparing user-defined lists to known biological association networks databases. Finally, the newly discovered biomarkers from the 3xTg-AD mice will be further validated, qualified, and ultimately used in conjunction with bioconjugated quantum dot biosensors to introduce a novel molecular diagnostic assay for the accurate, predictive, early, and pre-symptomatic detection of AD in the human population.

Introduction

Alzheimer's disease (AD) is a progressive neurodegenerative disorder characterized by global cognitive dysfunction (particularly memory loss), behavior or personality alterations, and impairments in the performance of the activities of daily living. The memory loss exhibited in AD is dependent on the hippocampal system, comprised of the dentate gyrus, cornu ammonis (Calif.)1-CA3, and rhinal cortices. As the disease progresses, global amnesia, dependent on other cortical areas, debilitates the individual (Hock and Lamb, 2001). AD is characterized by specific neuropathological alterations, including extracellular β-amyloid-containing (Aβ) plaques, intracellular neurofibrillary tangles (NFT) of abnormally phosphorylated tau (τ) protein, and degeneration of the cholinergic neurons in the basal forebrain (Auld et al., 2002). AD is recognized as the most prevalent dementia in mid-to-late life. It affects 7-10% of individuals over the age of 65 and an estimated 40% of persons over the age of 80. It is currently believed that AD affects over 4.5 million Americans and that 100,000 succumb to the disease annually with a projected 22 million individuals worldwide to develop dementia by the year 2025 (Crentsil, 2004). These figures are exacerbated by the fact that there is presently no reliable biomarker(s), that is, a characteristic that is objectively measured and evaluated as an indicator of normal biological processes, pathological processes, or pharmacological responses to a therapeutic intervention (Frank and Hargreaves, 2003), of AD. Thus, definitive diagnosis of AD is normally made upon autopsy, which prevents any new treatment efficacy to be extended overtime for the individual patient.

According to estimates used by the Alzheimer's Association and the National Institute on Aging, the national direct and indirect annual costs of caring for individuals with AD are at least $100 billion (Ernst and Hay, 1994). AD costs American business an estimated $61 billion a year of which $24.6 billion covers AD patient health care and $36.5 billion covers costs related to caregivers of individuals with the disease, including lost productivity, absenteeism, and worker replacement (Koppel, 2002). In addition, approximately half of all nursing home residents have AD or a related disorder with the average cost for nursing home care being $42,000 per year, but exceeding $70,000 per year in some areas of the United States (Rice, 1993). Finally, based on a 2001 report commissioned by the Alzheimer's Association, by 2010 Medicare costs for beneficiaries with AD are expected to increase 54.5% from $31.9 billion in 2000 to $49.3 billion, and Medicaid expenditures on residential dementia are expected to increase 80% from $18.2 billion to $33 billion in 2010 (Alzheimer's Association, 2001). Therefore, it is quite clear that, with the continued increase in the size of the aging population, AD remains and will be a major economic and social concern worldwide and a tremendous unmet medical need.

Modern DNA, RNA, and protein expression technologies are revolutionizing our view and understanding of current neurological diseases, such as AD, and enable researchers to analyze the concurrent expression patterns of very large numbers of genes. These new high-throughput genomic and proteomic technologies, collectively referred to as the Systems Biology approach (Ideker et al., 2001; Heath et al., 2003; Hood et al., 2004), such as DNA and protein microarrays, allow for the simultaneous study of thousands of genes and protein end products, and their alterations in regulation and modulation patterns in relation to disease state, time, and tissue specificity. However, due to post-transcriptional and post-translational (e.g., phosphorylation and glycolysation of proteins) modifications, the relationship between the level of mRNA and those of the protein end product is not always the same. In many instances, there is a positive correlation between the mRNA and protein levels in a tissue sample, but often there is no correlation, and frequently a negative correlation is observed (Lewandowski and Small, 2005). Thus, protein expression profiling is necessary as a follow-up procedure to any DNA microarray finding.

Advances in molecular genetic studies in the past two decades have allowed the identification of several genetic loci associated with AD. AD-related genes have been classified into genes with demonstrated mutations following a Mendelian inheritance pattern (i.e., mutational genetics), such as amyloid precursor protein (APP), presenilin-1 (PS1), and presenilin-2 (PS2), susceptibility genes or polymorphic loci potentially contributing to AD predisposition (i.e., susceptibility genetics), such as apolipoprotein E (APOE), alpha-2-macroglobulin (A2M), low density lipoprotein-related protein-1 (LRP1), interleukin-1 (IL1), and angiotensin I converting enzyme (ACE), and defective genes linked to mitochondrial DNA (mtDNA) with heteroplasmic transmission (reviewed in Cacabelos, 2002).

Recently, a triple-transgenic mouse model of AD (3xTg-AD) harboring three mutant genes, namely, β-amyloid precursor protein (βAPP_{Swe}), presenilin-1 (PS1_{M146V}), and tau_{P301}L, has been developed that uniquely expresses both of the hallmark neuropathological lesions associated with AD, that is, the Aβ plaques and NFT (Oddo et al., 2003a,b). These mice develop the Aβ and tau pathologies with a temporal- and regional-specific profile that closely resembles their development in the human AD brain. In fact, it has been observed that the extracellular β-amyloid deposits initiate in the cerebral cortex and with aging progress to the hippocampus, whereas the tau pathology first appear in the hippocampus and progress to the cortex (Oddo et al., 2003a,b). This observed pattern of Aβ deposition developing prior to tau pathology is also consistent with the current widely accepted amyloid cascade hypothesis of AD.

According to a recent study (Billings et al., 2005), at 2-months old, the pre-pathologic 3xTg-AD mice are cognitively normal. However, at 4-months, the mice manifest the earliest cognitive impairment as a deficit in long-term retention, which correlates with the accumulation of intraneuronal Aβ in the hippocampus and amygdala. The results from this study strongly suggest the intraneuronal accumulation of Aβ in the onset of cognitive dysfunction in the 3xTg-AD mice. On the other hand, the earliest sign of tau pathology in the 3xTg-AD mice appears with the accumulation of tau in the somatodendritic compartment at 6-months of age. The tangle pathology is quite advanced and apparent by 18 to 20-months with different silver stains and immunoreactive with several phospho-specific tau antibodies, such as AT8 and PHF-1 (Oddo et al., 2003b; Oddo et al., 2005).

In addition, since the 3xTg-AD mice are generated from simultaneously microinjecting two transgenes (i.e., βAPP and tau) into single-cell embryos from homozygous PS1_{M146v} knock-in mice, rather than crossing independent lines, the mice all have the same genetic background.

Compared to crossbreeding, this approach offers several major advantages. For example, deriving a large colony of the 3xTg-AD is straightforward, cost-effective, and does not require extensive genotyping of the progeny. Moreover, the easy propagation of this transgenic line facilitates their crossing to other transgenic or gene-targeted mice to assess the impact of other genotypes on the neuropathological or physiological phenotype. Finally, since multiple transgenes are introduced into an animal without altering or mixing the background genetic constitution, an important confounding variable is avoided. This provides crucial parameter control for behavioral, genomic, proteomic, and vaccine-based experiments.

Furthermore, given that transgenic mouse models of AD can be scientifically controlled with great precision in comparison with human research efforts, biomarker discovery in the 3xTg-AD mice can progress rather quickly. Such biomarker discovery endeavors via a transgenic mouse model of the disease can greatly facilitate the process of drug discovery and development and early detection of AD in humans. For example, potential molecular markers of AD elucidated through examination of the 3xTg-AD mice can be immediately validated by cross-comparison with peer-reviewed, published human data, and subsequently by directly assaying representative human brain tissue. There are numerous efforts presently underway to make such data readily available. For instance, a large-scale project to characterize human AD tissue using Affymetrix gene expression microarrays is currently being carried out through a coordinated effort of numerous academic laboratories worldwide. The preliminary data from these endeavors are expected to be published in early 2006. In addition to large-scale high-throughput functional genomic studies like these to corroborate data from transgenic mouse models in humans, it is possible to access tissue from the various brain banks storing human AD tissue (www.alzforum.org) for focused mouse biomarker validation studies, such as with western blot, ELISA, two-dimensional gel electrophoresis (2-D gel), mass spectrometry (MS), and functional protein microarrays (e.g., Invitrogen's ProtoArrays).

In the present study, we will examine the 3xTg-AD mice not only with phenotypic assessment, namely, immunohistochemical and behavioral measures, but also with genomic (i.e., DNA microarrays), relative quantitative (i.e., iTRAQ coupled with MudPIT) and functional proteomic (i.e., ProtoArrays), and bioinformatic (i.e., GeneGo's MetaCore integrated software suite) methods. Representative tissue samples taken from frontal cortical and hippocampal areas, particularly the entorhinal cortex (EC), of each mouse will be used for immunohistochemical, gene, and protein expression analysis at specific time points [i.e., 2 (pre-pathological), 6 (initial pathology), 15 (tangle pathology), and 24 (full pathology) months of age]. The EC has been found to be the hippocampal subregion that is differentially targeted by early AD rather than normal aging in several species, including rodents (Small et al., 2004). In addition, quantitative real-time reverse transcription-polymerase chain reaction (RT-PCR) analysis will be used to confirm the genomic findings. Therefore, to our knowledge, the present study will be the first of its kind to perform a comprehensive Systems Biology analysis of the 3xTg-AD mice to identify novel biomarkers associated with AD. These newly discovered and validated sets of biomarkers will in turn be used to introduce the more short-term development of a novel molecular diagnostic assay, involving the use of bioconjugated quantum dot (QD) nanocrystals or probes, for the highly sensitive, predictive, reliable, accurate, and pre-symptomatic detection of AD in the human population and as the basis for the eventual long-term development of new therapeutic agents for the disease. These endeavors will hopefully enable the ultimate development of personalized medicines (i.e., pharmacogenomics) for individuals within the AD population worldwide.

Research Design and Methods

[image]

Experimental Design

The 3xTg-AD mice will be compared with age, gender, and genetic background-matched nontransgenic controls at four different time points, namely, 2 (pre-pathological), 6 (initial pathology), 15 (tangle pathology), and 24 (full pathology) months of age. For gene and protein expression analysis, at least three mice will be used for each time point and each control group. In the case of gene expression analysis, mRNA will be extracted from each mouse from pre-dissected hippocampal, particularly the entorhinal cortex (EC), and frontal-cortical regions, and individually analyzed using Affymetrix mouse genome expression microarrays for individual sample resolution of gene expression; cerebellar subregions will also be analyzed as negative controls. In addition, quantitative real-time reverse transcription-polymerase chain reaction (RT-PCR) analysis will be used to confirm the DNA microarray findings. For protein expression profiling and analysis (i.e., identification and relative quantification), the technique of isobaric Tagging for Relative and Absolute protein Quantification (iTRAQ) combined with Multidimensional Protein Identification Technology (MudPIT) will be performed on cell lysates from hippocampal and frontal cortical samples of 3xTg-AD and control mice at each time point. In addition, candidate proteins will be chosen based on significantly differing expression levels from 3xTg-AD versus control brain tissue samples and further analyzed for functional parameters, such as protein-protein interactions, via Invitrogen's ProtoArray technology. Bioinformatic analysis of gene and protein expression data will subsequently be used to yield biomarker candidates. Finally, phenotypic analysis, such as immunohistochemical and behavioral measures, will be concurrently performed throughout the study to monitor the progress of disease pathology and behavioral alterations at each time point.

Mice and Surgical Procedures

Similar to the methodology described in Oddo et al. (2003a,b), 3xTg-AD mice harboring APP_{Swe}, PS1_{M146V}, and tau_{P301L} transgenes, which were generated by simultaneous microinjection of two independent transgene constructs encoding human APP_{Swe} (i.e., Swedish familial mutation) and tau_{P301L} into the pronuclei of single-cell embryos harvested from mutant homozygous PS1_{M146V} knockin mice (see FIG. 2). The PS1 knockin mice were originally generated on a hybrid 129/C57BL/6 background (Guo et al., 1999). Southern blot analysis of tail DNA will subsequently be used to identify the transgenic mice (LaFerla et al., 1995; Sugarman et al., 2002).

[image]

Spontaneous Alternation Y Maze Task. As previously described (Holcomb et al., 1998), this learning paradigm involves hippocampal circuits that direct spatial working memory and bypasses the need for any training, reward, or punishment. The Y maze apparatus is comprised of three acrylic arms at 120° angles to one another (see FIG. 3). The dimensions of each arm are as follows: 40 cm length, 17 cm height, and 4 cm width at the bottom and 13 cm width at the top. Each mouse, being placed in the center of the maze, will be given 8 minutes to navigate through the maze freely. The sequence of entry and number of maze arms entered (entry defined as having all hind paws within the arm) will be recorded. Percentage alternation will be calculated as described in the above-mentioned literature.

Spatial Reference Morris Water Maze (MWM) Training. Mice will be trained to swim to a submerged (and functionally invisible) 14 cm diameter circular clear Plexiglas platform (see FIG. 3). After being released from one randomly selected start point (of 4 designated start points), mice will be allowed 60 sec to locate and escape onto the platform, after which they will be manually guided to the platform on which they will remain for 10 sec. During the inter-trial interval, the mice will be placed under a warming lamp in a holding cage for 25 sec. All mice will be trained to criterion (<20 sec mean escape latency) to control for memory differences due to lack of task learning. Cued platform training will be utilized to control for visual ability and intact striatal-mediated learning (Billings et al., 2005). This will consist of four consecutive trials daily in which starting position (along edge of tank) and platform location will be altered for each trial.

Retention of the spatial reference training will be measured at 1.5 hr and at 24 hr post last training trial, consisting of a 60 sec free swim without platform (following Billings et al., 2005). The parameters assessed during the retention trials will include initial latency to cross the platform location, number of platform location crosses, and time spent in quadrant opposite platform.

[image]

[image]

ELISA Quantitation of Brain Aβ Levels

After behavioral tests are completed, the Aβ₁₋₄₀ and Aβ₁₋₄₂ levels will be measured using a sensitive sandwich enzyme-linked immunosorbent assay (ELISA) system (Duff et al., 1996; Miller et al., 2003). Frozen hemibrains will be extracted in 0.2% diethlyamine with 50 nM NaCl and centrifuged at 20,000×g for 1 hr at 4° C. to remove insoluble material. The resulting supernatant fractions will be analyzed using the well-known BNT77/BA27 and BNT77/BC05 antibody systems to detect Aβ₁₋₄₀ and Aβ₁₋₄₂, respectively. These sandwich ELISAs are known to recognize both human and mouse Aβ₁₋₄₀ and Aβ₁₋₄₂ with equivalent sensitivities.

Immunohistochemistry

Initial processing will follow the protocol as previously described (Oddo et al., 2003a,b; Billings et al., 2005). Briefly, mice will be sacrificed by CO₂ asphyxiation, and the brains will be rapidly removed and fixed for 48 hr in 4% paraformaldehyde. Free-floating (5 μm thick) sections will be mounted onto silane-coated slides and processed with the following antibodies: anti-Aβ 6E10 and 4G8 (Signet Laboratories, Dedham, Mass.), anti-Aβ 1560 (Chemicon), A11 (Kayed et al., 2003), anti-APP 22C11 (Chemicon), anti-Tau HT7, AT8, AT180 (Innogenetics), Tau C17 (Santa Cruz), Tau 5 (Calbiochem), anti-GFAP (Dako), and anti-actin (Sigma). Primary antibodies will be applied at dilutions of 1:3000 for GFAP; 1:1000 for 6E10; 1:500 for 1560, AT8, AT180, and Tau 5; and 1:200 for HT7. Sections will then be developed with diaminobenzidine (DAB) substrate using the avidin-biotin horseradish peroxidase system (Vector Labs).

Confocal Microscopy

To achieve highly resolved target-specific images, a two-way fluorescent immunolabeling technique, involving application of a primary antibody followed by a fluorescent secondary antibody, will be implemented. After initial immunohistochemical processing, tissue will be incubated for 1 hr in fluorescently labeled anti-mouse 2° antibody (Alexa 488; 1:200; Molecular Probes Inc., Eugene, Oreg.). Slices will then be incubated for 20 min in TOTO-13 iodide to add nuclear markers (Molecular Probes Inc.; 1:200 in PBS). Confocal images will subsequently be captured on an MRC 1024 (BioRad, Hercules, Calif.) confocal system.

Tissue Extraction and RNA Isolation

Mice will be sacrificed by decapitation; their brains will be rapidly extracted whole, and immediately submerged into a microdissection well containing RNALater (Ambion, Austin, Tex.) buffer to maximize integrity of total RNA. Hippocampi, frontal cortices, and cerebellum are microdissected and stored separately for 24 hours at 4° C. in RNALater buffer to allow for optimal cellular penetration. Tissue is then removed from buffer and stored dry at −20° C. for 2-6 weeks, or up to 6 months at −80° C., until ready for RNA isolation.

In addition, total RNA from representative hippocampal and frontal cortical areas of mice will be extracted using the TRIzol reagent according to the manufacturer's specifications (Invitrogen, Carlsbad, Calif.). Samples will be first homogenized in TRIzol reagent for 10 sec. After mixing with chloroform, the samples will then be centrifuged for 15 min at 12,000×g at 4° C. Isopropanol will subsequently be added to the aqueous phase for RNA precipitation. This precipitation mix will be centrifuged for 10 min at 12,000×g at 4° C. The RNA pellet will then be washed once with cold 75% ethanol and briefly air dried in an RNase-free hood. Total RNA will subsequently be resuspended in nuclease-free water and subjected to TURBO DNase treatment (Ambion, Austin, Tex.). Genomic DNA-free RNA will be further purified by an RNeasy column (Qiagen, Valencia, Calif.). Finally, RNA will be eluted from the column using nuclease-free low-pH sodium citrate storage buffer.

Laser Capture Microdissection (LCM) of Tissue Sections

To obtain homogenous populations of cells from heterogeneous hippocampal and frontal cortical tissue sections, the method of laser capture microdissection (LCM) will be employed. Selected fresh-frozen areas of the hippocampus and frontal cortex will be sectioned at 6 μm thick, briefly fixed with acetone, and Nissl stained for neuronal identification based on neuronal morphology and cytoarchitecture. Once a region of interest is selected in the stained section, an Arcturus PixCell I laser capture microscope (Arcturus Engineering, Mountain View, Calif.) with a beam size of 30 μm, which is sufficient to capture cell clusters containing as few as 20 cells, will be used to capture particular neuron populations. In addition, a Leica LS-AMD (Leica Microsystems Bannockburn, IL) outfitted with fluorescent optics and a minimum beam width of less than 1 μm will be used for selective visualization and capture of stained neurons. The dissected tissue will then be transferred to a plastic membrane (“cap”; Arcturus Engineering) and recovered in a microcentrifuge tube for subsequent nucleic acid extraction and microarray analysis. Tissue contaminants will be removed from the transfer caps with Arcturus's CapSure sticky pads. All procedures will be performed under RNAse free conditions.

DNA Microarray Hybridization and Analysis

The Affymetrix Mouse Genome 430 2.0 Arrays (Affymetrix, Santa Clara, Calif.) that contain 45,101 probe sets will be used in the present study to examine gene expression patterns. Following isolation of total RNA (mRNA) from hippocampal and frontal cortical brain tissue from each animal, all subsequent technical procedure, including quality control of RNA, labeling with biotin-rNTPs, hybridization, and scanning of the arrays will be performed in the DNA Array Core Facility at the University of California, Irvine (UCI) under the supervision of Denis Heck, Ph.D.

For subsequent DNA microarray data analysis, the GeneSpring GX (Agilent Technologies, Palo Alto, Calif.) software will be used to procure statistically significant and disease relevant gene lists from Affymetrix data image files (see FIG. 4). Briefly, data passes through pre-processing, normalization, quality control (QC), and statistical measure (i.e., t-test or ANOVA) filters to develop highly relevant gene lists. Pre-processing measures analyze the biotinylated detection grids to eliminate faulty signals and outliers, and score detection grids for raw signal intensities. Normalization filters establish per-chip normalization of total intensity, and per-gene normalization across samples in order to make data from large replicate cohorts relevant to one-another. A cross-gene error model is calculated and base/proportional measurements give control signals for each gene. QC filters, such as filtering of genes by control signal cut-offs, creates gene lists that have reliable signal intensities relative to one another. These gene lists can be further filtered by fold change to eliminate fold changes that are unlikely to yield meaningful changes (e.g., <1.2-1.5 fold differences). A host of statistical filters ranging in stringency are then implemented for pairwise comparisons or multifactor one-way ANOVA tests to procure statistically relevant disease-related gene lists. These gene lists are then incorporated into bioinformatic meta-analysis for development of novel molecular networks of disease-related biomarkers.

[image]

Protein Expression Profiling Using iTRAQ Coupled with MudPIT

To determine the relative quantitative protein expression profiles of brain tissue samples from the hippocampal and frontal cortical areas of 3xTg-AD mice as compared to controls, the techniques of isobaric Tagging for Relative and Absolute protein Quantification (iTRAQ, Applied Biosystems, Foster City, Calif.) coupled with Multidimensional Protein Identification Technology (MudPIT) will be performed (DeSouza et al., 2005) (see FIGS. 5and 6).

[image]

[image]

Protein extracts (approximately 150 mg) from up to 4 different experimental and control groups (2 shown on chart from FIG. 5) are reduced, alkylated, and digested with trypsin in an amine-free buffer system, in parallel (Ross et al., 2004). The resulting peptides are then labeled with the iTRAQ Reagents for 1 hr at room temperature. Upon completion of labeling, the samples are combined and directly analyzed by 2-dimensional High Performance Liquid Chromatography (2D HPLC), including separation by strong cation exchange (SCX) coupled with fused silica capillaries and reverse phase chromatography (RP) for optimal peptide separation. An LC Packings UltiMate LC system (Dionex) will be used to analyze array-plated samples that is interfaced offline onto a 4700 Proteomics Analyzer (Sciex/Appplied Biosystems) (Ross et al., 2004). Spectra from the 4700 Proteomics Analyzer will be loaded into the GPS Explorer software (Applied Biosystems) and searched against a murine protein database with trypsin specificity using the MASCOT search engine (www.matrixscience.com) (Zhang et al., 2005). Data will be normalized to the vehicle-treated control values for comparison with experimental groups. To allow for the identification of potentially unlabelled peptides, protein database searches will also be performed with the iTRAQ Reagent derivatives as variable modifications. Finally, a paired, two-tailed Student's t-test will be performed for statistical analysis of the data.

Functional Proteomics Using ProtoArrays

In order to characterize specific protein interactions of differentially expressed proteins identified from the quantitative proteomic analysis, human ProtoArray protein microarrays (Invitrogen, Carlsbad, Calif.) will be employed to screen labeled probes against over 5,000 uniques proteins (see FIG. 7). Functional protein microarrays present an important new tool ideally suited to the mapping of biological pathways. Protein microarrays were developed to provide miniaturized high-throughput tools to study protein function, expression and post-translational modifications. Functional protein microarrays can be used to reproduce most major types of interactions and enzymatic activities seen in biochemical pathways. Because of the unique ability to address different aspects of biological pathways, functional protein microarray technology is primed to make significant contributions to the understanding of disease pathways for both basic and drug research (Predki et al., 2004; Merkel et al., 2005). All proteins are expressed in a baculovirus system to maintain post-translational modifications, and purified under native conditions to preserve maximum functionality and protein structure. Briefly, human protein microarrays containing over 5,000 full-length proteins will be screened with a probe containing single V5 or biotin tags. Interacting proteins are then detected using AlexaFluor labeled anti-V5 antibody or Alexa Fluor labeled streptavidin. Data analysis can be performed using a manual analysis of the arrays to identify significant signals on the slide. However, it is recommended to use a software program, such as the ProtoArray Prospector (Invitrogen, Carlsbad, Calif.), for analysis of protein-protein interaction data on Invitrogen Protoarrays. This is a freeware tool that quickly identifies statistically significant signals on the arrays. To date, approximately 80% of interactions that are detected by a solution-based assay (gel mobility shift) are also observed on the ProtoArrays.

In addition, it is not impractical to run mouse candidate biomarkers against human protein microarrays since the 3xTg-AD mice contain human transgene variants (Oddo et al., 2003a, b), and presumably, alternative splicing is highly conserved and exon sequence homology among mice and humans is quite strong (Sugnet et al., 2004; Thanaraj et al., 2003). Thus, candidate molecules, such as proteins identified by the abovementioned iTRAQ combined with MudPIT analysis of 3xTg-AD versus control brain tissue, or molecules from other sources, can be hybridized to human ProtoArray protein microarrays to attempt to reveal novel protein-molecule interactions. This information can then be used to further map biochemical pathways in disease pathogenesis, potentially uncovering additional novel disease biomarkers.

[image]

Statistical Analysis

Cyber-T statistical analysis software will be used for multifactor ANOVA (ANalaysis Of VAriance), including genotype and age, will be used to analyze behavior scores of the mice. Post hoc Fisher's PLSD tests will be performed to determine significance of differences between the groups when appropriate. In addition, the immunohistochemistry scores will be analyzed by ANOVA with results being considered significant when p<0.05.

Statistical significance in microarray analysis will be carried out by the GeneSpring GX one-way ANOVA measures. Briefly, a parameter (e.g., disease state) is compared across experimental groups by one of a host of algorithms, such as a Welch test, Student's t-test, or non-parametric test, and set to a desired p-value (e.g., 0.05). A multiple testing correction may be applied, and a post hoc test can be used in conjunction with ANOVA to determine which specific group pairs are statistically different from each other.

Discovery of Novel Molecular Networks

Differentially expressed genes and proteins, identified by Agilent GeneSpring GX software and quantitative protein expression/functional proteomic data, respectively, will be further analyzed by MetaCore (GeneGo, St Joseph, Mich.). MetaCore is a platform that has the largest Systems Biology proprietary manually curated database with a suite of software tools for analysis. GeneGo uses Ph.D. level annotators that are employees to read full text articles to populate the database with genes, proteins, hormones, compounds, metabolites and transcriptional factors, 15 mechanisms of interaction, direction, and links to papers. MetaCore has the unique ability to provide merged metabolic and signaling pathway networks as well as a metabolic parser for visualizing MS concentration data in the context of canonical maps and pathways. Furthermore, MetaCore has the ability to concurrently visualize gene expression and proteomics data as well as multiple time points, dosages, and treatments to identify key functions and pathways that distinguish biological states. Deeper analysis can be completed by working with tissue, subcellular localization, interaction, ortholog, and functional process filters as well as understanding other drug targets in the networks. MetaCore can also build disease specific signature networks as a starting point for investigation (see FIG. 8).

The range of functionality of MetaCore is extensive. Initially, prospective annotated gene lists (via GeneSpring GX) or protein lists (via quantitative proteomics analysis) are uploaded into the MetaCore data manager. The MetaLink add-on feature can integrate protein interaction lists acquired by Protoarray Prospector functional analysis. These lists of genes, proteins, and protein-protein interactions are fully integratable, and can be cross-filtered against a proprietary database of canonical maps, which feature hundreds of discrete functional biological processes and their relevant Gene Ontology (GO) processes. Resulting processes on the list of statistically relevant functional maps are then annotated with the ratio of biomarkers present over total for each specific process, and the p-value of specificity of interaction calculated based on hypergeometric distribution. When visualized, these maps give valuable insight into fundamental differential patterns of metabolite and signal processing, such as within the brain regions of the 3xTg-AD mice. The maps can be visualized, combined and/or exported in conjunction with complementary maps or imported biomarker lists. These lists of biomarkers can also be compared directly using a host of “logical operator” processing filters to create interaction lists. Both lists and maps can be mined for distribution of relevant GO processes, and biomarker lists can be further refined before novel regulatory networks are then built around these lists (see FIG. 9).

Novel networks comprise of an expanded manually curated interactome of well-established (i.e., collected from top-tier peer-reviewed literature) as well as more putative interactions, beyond the scope of the canonical maps. A novel regulatory and/or metabolic network can be built around specific lists of functional biomarkers by virtue of a variety of parsing algorithms, ranging from direct interactions to complex functional networks. The resulting novel networks can be either manually mined for novel processes, or preprogrammed mining functions can extract existing patterns of functional or disease processes, GO processes, filtered by cell/tissue type, subcellular localization and/or mined for ancillary, unmapped interactions. The “orthologs” function allows regulatory networks built around one species to be translated to another species, such as a novel network of biomarkers built around a mouse domain that can be translated to homologous processes in the human domain of molecular interactions. Biomarker lists are extracted from whole or partial novel networks of biomarker interactions that can further be implemented into developing highly sensitive and specific disease detection assays and therapeutic agents.

[image]

[image]

Validation and Qualification of Early Detection Biomarkers

The translational efficacy of novel biomarkers in early detection diagnosis should be rigorously ascertained. To this end a battery of genomic and proteomic assays of the panel of biomarkers will be paired with statistical measures to determine which, if any, of the identified panel of biomarkers are actually predictive of development of AD, and whether there is a specific combination of biomarkers within the panel that maximizes disease prediction sensitivity and specificity.

Part 1: Quantitative Real-Time RT-PCR

Quantitative real-time reverse transcription-polymerase chain reaction (RT-PCR) will be performed as described elsewhere (Saura et al., 2004), to validate the genomically-derived biomarkers. Briefly, part of the RNA samples used for the microarray studies will be treated with DNase I and reverse transcribed in the presence of random hexamers. PCR reactions will be performed using SYBR Green PCR Master Mix in an ABI PRISM 7700 Sequence Detector (Applied Biosystems) with 10 μl of diluted (1:25) CDNA and gene-specific primers. Reactions will be performed in duplicate and the threshold cycle values normalized to 18 S RNA. Electrophoresis will then be used to confirm the correct sizes of the PCR products. Alternatively, a melting curve of each PCR reaction will be generated to verify a single specific PCR product.

Part 2: Multi-Analyte Profiling

A quantitative and precise Multi-Analyte Profile (MAP) test employing customized fluorescently-encoded microsphere-based sandwich-ELISA assay technology (Charles River Laboratories, Wilmington, Mass., in conjunction with Rules-Based Medicine, Austin, Tex.) will be designed combining relevant antibodies for quantitative detection of protein levels for the panel of novel biomarkers as well as other suspected biomarkers of AD selected from current literature, such as specific isoprostanes, tau, Aβ, sulfatide, homocysteine, and others. The 3xTg-AD mice fluids (e.g., blood, urine and CSF) at various time points will subsequently be examined in parallel with human fluid samples acquired from the Institute for Brain Aging and Dementia (IBAD) at the University of California, Irvine.

In addition, predictive surrogate biomarkers of AD will be identified by supervised and unsupervised statistical clustering methods applied to training and validation sample sets (e.g., Wang et al., 2005). First, protein levels across mouse samples are normalized and deviation relative to mean calculated. For training sets, a relativity plot is calculated to cluster similar data points in close proximity (e.g., K-nearest neighbor unsupervised clustering). A standard t-test will be employed to determine statistically significant markers, which demonstrate sufficient dissimilarity between control and disease groups. Finally, a qualification sample set will be assayed, in which supervised clustering analysis (e.g., class prediction using weighted voting schema) will evaluate the predictive value of statistically significant biomarkers from the training set to determine sensitivity and specificity data for selected markers.

Bioconjugated Quantum Dot (QD) Nanocrystals as a Novel Molecular Diagnostic Tool

The newly identified and validated biomarkers from analyses of the gene and protein interaction networks of the 3xTg-AD mice versus controls will be used in conjunction with bioconjugated quantum dot (QD) nanocrystals to screen for the presence of AD specific biomarkers in samples of readily accessible bodily fluids, such as blood, urine, saliva, tears, and/or cerebrospinal fluid (CSF), of individuals. The current gold standards for detecting low copy-number nucleic acids and proteins in bodily fluids are PCR combined with a variety of molecular fluorophore assays and enzyme-linked immunosorbent assay (ELISA), respectively. However, the clinical use of these assays are labor intensive, time consuming, prohibitive of multiplexing, and expensive. Bioconjugated QD probes, which are linked to biological molecules like monoclonal antibodies, peptides, proteins, or nucleic acids and contain bright and stable fluorescent light emission and multiplexing potential (i.e., capability to detect multiple disease markers simultaneously), provide a novel highly sensitive approach to detect low-abundant copy numbers of potential disease biomarkers (e.g., nucleic acids and proteins) in bodily fluid and tissue samples (see FIG. 10). QDs with their intrinsic high spatial resolution and sensitivity of fluorescence imaging can not only serve as sensitive probes for disease biomarkers, but they could also enable the detection of hundreds to thousands of simultaneously (i.e., multiplexing; Smith et al., 2006a, b).

In addition, bioconjugated QD probes can be used as high-resolution contrast makers for medical imaging tools, such as functional magnetic resonance imaging (fMRI) and positron emission tomography (PET), to noninvasively detect for the early presence of AD specific biomarkers within brain regions in vivo, such as the hippocampus and frontal cortex, of individuals at risk and susceptible of developing AD (Smith et al., 2006a, b).

[image]

### REFERENCES

Alzheimer's Association. 2001. Medicare and Medicaid costs for people with Alzheimer's disease. Washington, D.C.: The Lewin Group. p. 1.
Auld, D. S., Kornecook, T. J., Bastianetto, S., and Quirion, R. 2002. Alzheimer's disease and the basal forebrain cholinergic system: relations to beta-amyloid peptides, cognition, and treatment strategies. Progress in Neurobiology. 68:209-245.
Billings, L. M., Oddo, S., Green, K. N., McGaugh, J. L., and LaFerla, F. M. 2005. Intraneuronal Aβ causes the onset of early Alzheimer's disease-related cognitive deficits in transgenic mice. Neuron. 45:675-688.
Cacabelos, R. 2002. Pharmacogenomics in Alzheimer's disease. Mini Reviews in Medicinal Chemistry. 2(1):59-84.
Crentsil, V. 2004. The pharmacogenomics of Alzheimer's disease. Ageing Research Reviews. 3(2):153-169.
DeSouza, L., Diehl G., Rodrigues, M. J., Guo, J., Romaschin, A. D., Colgan, T. J., and Siu, K. W. 2005. Search for cancer markers from endometrial tissues using differentially labeled tags iTRAQ and cICAT with multidimensional liquid chromatography and tandem mass spectrometry. Journal of Proteome Research. 4:377-386.
Duff, K., Eckman, C., Zehr, C., Yu, X., Prada, C. M., Perez-tur, J., Hutton, M., Buee, L., Harigaya, Y., Yager, D., et al. 1996. Increased amyloid-beta42(43) in brains of mice expressing mutant presenilin 1. Nature. 383:710-713.
Ernst, R. L. and Hay, J. W. 1994. The U.S. economic and social costs of Alzheimer's disease revisited. American Journal of Public Health. 84(8):1261-1264.
Guo, Q., Fu, W., Sopher, B. L., Miller, M. W., Ware, C. B., Martin, G. M., and Mattson, M. P. 1999. Increased vulnerability of hippocampal neurons to excitotoxic necrosis in presenilin-1 mutant knock-in mice. Nature Medicine. 5:101-106.
Hardy, J. and Selkoe, D. J. 2002. The amyloid hypothesis of Alzheimer's disease: progress and problems on the road to therapeutics. Science. 297:253-256.
Heath, J. R., Phelps, M. E., and Hood, L. 2003. NanoSystems Biology. Molecular Imaging and Biology. 5(5):312-325.
Hock, B. J. and Lamb, B. T. 2001. Transgenic mouse models of Alzheimer's disease. Trends in Genetics. 17(10):S7-S12.
Holcomb, L., Gordon, M. N., McGowan, E., Yu, X., Benkovic, S., Jantzen, P., Wright, K., Saad, I., Mueller, R., Morgan, D., Sanders, S., Zehr, C., O'Campo, K., Hardy, J., Prada, C. M., Eckman, C., Younkin, S., Hsiao, K., and Duff, K. 1998. Accelerated Alzheimer-type phenotype in transgenic mice carrying both mutant amyloid precursor protein and presenilin 1 transgenes. Nature Medicine. 4:97-100.
Hood, L., Heath, J. R., Phelps, M. E., and Biaoyang, L. 2004. Systems Biology and new technologies enable predictive and preventative medicine. Science. 306:640-643.
Ideker, T., Galitski, T., and Hood, L. 2001. A new approach to decoding life: Systems Biology. Annual Review of Genomics and Human Geneties. 2:343-372.
Kayed, R., Head, E., Thompson, J. L., McIntire, T. M., Milton, S. C., Cotman, C. W., and Glabe, C. G. 2003. Common structure of soluble amyloid oligomers implies common mechanism of pathogenesis. Science. 300:486-489.
Koppel, R. 2002. Alzheimer's Disease: The costs to U.S. businesses in 2002. Washington, D.C.: Alzheimer's Association.
LaFerla, F. M., Tinkle, B. T., Bieberich, C. J., Haudenschild, C. C., and Jay, G. 1995. The Alzheimer's A beta peptide induces neurodegeneration and apoptotic cell death in transgenic mice. Nature Genetics. 9:21-30.
Lewandowski, N. M. and Small, S. A. 2005. Brain Microarray: Finding Needles in Molecular Haystacks. Journal of Neuroscience. 25(45):10341-10346.
Merkel, J. S., Michaud, G. A., Salcius, M., Barry Schweitzer, B., and Predki, P. F. 2005. Functional protein microarrays: just how functional are they? Current Opinion in Biotechnology. 16:447-452
Miller, B. C., Eckman, E. A., Sambamurti, K., Dobbs, N., Chow, K. M., Eckman, C. B., Hersh, L. B., and Thiele, D. L. 2003. Amyloid-β-peptide levels in brain are inversely correlated with insulysin activity levels in vivo. Proceedings of the National Academy of Sciences, USA. 100:6221-6226.
Nikilsky, Y., Nikolskaya, T., and Bugrim, A. 2005. Biological networks and analysis of experimental data in drug discovery. Drug Discovery Today. 10(9):653-662.
Oddo, S., Caccamo, A., Kitazawa, M., Tseng, B. P., and LaFerla, F. M. 2003a. Amyloid depositon precedes tangle formation in a triple transgenic model of Alzheimer's disease. Neurobiology of Aging. 24:1063-1070.
Oddo, S., Caccamo, A., Shepherd, J. D., Murphy, M. P., Golde, T. E., Kayed, R., Metherate, R., Mattson, M. P., Akbari, Y., and LaFerla, F. M. 2003b. Triple-transgenic model of Alzheimer's disease with plaques and tangles: intracellular Aβ and synaptic dysfunction. Neuron. 39:409-421.
Predki, P. F. 2004. Functional protein microarrays: ripe for discovery. Current Opinion in Chemical Biology. 8(1):8-13.
Rice, D. P., Fox, P. J., Max, W., Webber, P. A., Lindeman, D. A., Hauck, W. W., and Segura, E. 1993. The economic burden of Alzheimer's disease. Health Affairs. 12(2):164-176.
Ross, P. L., Huang, Y. N., Marchese, J. N., Williamson, B., Parker, K., Hattan, S., Khainovski, N., Pillai, S., Dey, S., Daniels, S., Pukayastha, S., Jubasz, P., Martin, S., Bartlet-Jones, M., He, F., Jacobson, A., and Pappin, D. J. 2004. Multiplexed protein quantitation in Saccharomyces cerevisiae using amine-reactive isobaric tagging reagents. Molecular and Cellular Proteomics. 3:1154-1169.
Saura, C. A., Choi, S. Y., Beglopoulos, V., Malkani, S., Zhang, D., Shankaranarayana Rao, B. S., Chattarji, S., Kelleher, R. J., Kandel, E. R., Duff. K., Kirkwood, A., and Shen, J. 2004. Loss of presenilin function causes impairments of memory and synaptic plasticity followed by age-dependent neurodegeneration. Neuron. 42:23-36.
Selkoe, D. J. and Schenk, D. 2003. Alzheimer's disease: molecular understanding predicts amyloid-based therapeutics. Annual Review of Pharmacology and Toxicology. 43:545-584.
Small, S. A., Chawla, M. K., Buonocore, M., Rapp, P. R., and Barnes, C. A. 2004. Imaging correlates of brain function in monkeys and rats isolates a hippocampal subregion differentially vulnerable to aging. Proceedings of the National Academy of Sciences, USA. 101(18):7181-7186.
Smith, A. M., Dave, S., Nie, S., True, L., and Gao, X. 2006a. Multicolor quantum dots for molecular diagnostics of cancer. Expert Review in Molecular Diagnostics. 6(2):231-244.
Smith, A. M., Ruan, G., Rhyner, M. N., and Nie, S. 2006b. Engineering luminescent quantum dots for in vivo molecular and cellular imaging. Annals of Biomedical Engineering. 1-12.
Sugarman, M. C., Yamasaki, T. R., Oddo, S., Echegoyen, J. C., Murphy, M. P., Golde, T. E., Jannatipour, M., Leissring, M. A., and LaFerla, F. M. 2002. Inclusion body myositis-like phenotype induced by transgenic overexpression of beta APP in skeletal muscle. Proceedings of the National Academy of Sciences, USA. 99:6334-6339.
Sugnet, C. W., Kent, W. J., Ares, M. Jr., and Haussler, D. 2004. Transcriptome and genome conservation of alternative splicing events in humans and mice. Pacific Symposium on Biocomputing 2004. 66-77.
Thanaraj, T. A., Clark, F., and Muilu, J. 2003. Conservation of human alternative splice events in mouse. Nucleic Acids Research. 31(10):2544-2552.
Wang, X., Yu, J., Sreekumar, A., Varambally, S., Shen, R., Giacherio, D., Mehra, R., Montie, J. E., Pienta, K. J., Sanda, M. G., Kantoff, P. W., Rubin, M. A., Wei, J. T., Ghosh, D., and Chinnaiyan, A. M. 2005. Autoantibody signatures in prostate cancer. New England Journal of Medicine. 353:1224-1235.
Wu, Z. and Irizarry, R. A. 2004. Stochastic Models Inspired by Hybridization Theory for Short Oligonucleotide Arrays. Proceedings of the 8th International Conference on Computational Molecular Biology. RECOMB.
Zhang, Y., Wolf-Yadlin, A., Ross, P. L., Pappin, D. J., Rush, J., Lauffenburger, D. A., and White, F. M. 2005. Time-resolved mass spectrometry of tyrosine phosphorylation sites in the epidermal growth factor receptor signaling network reveals dynamic modules. Molecular and Cellular Proteomics. 4:1240-1250.

1. A method for the identification of biomarkers for Alzheimer's disease (AD) comprising, in combination:
providing a plurality of 3xTg-AD mice and control mice;
at each of a plurality of time points, subjecting the 3xTg-AD mice and the control mice to at least one assay producing a candidate set of biomarkers for the progression of AD, whereby the candidate set of biomarkers for the progression of AD is determined by comparing data derived from the at least one assay in 3xTg-AD mice to the data derived from the control mice; and
determining whether the candidate biomarkers are predictive of the progression of AD.

2. The method of claim 1, wherein the assay is a behavioral assay of the 3xTg-AD mice and the control mice.

3. The method of claim 1, further comprising at each time point, quantifying the levels of at least one of β-amyloid containing plaques, intracellular neurofibrillary tangles, and abnormally phosphorylated tau protein.

4. The method of claim 3, wherein Aβ₁₋₄₀ and Aβ₁₋₄₂ is quantified in the 3xTg-AD mice and the control mice.

5. The method of claim 4, wherein an enzyme-linked immunosorbant assay is used in conjunction with the quantification of Aβ₁₋₄₀ and Aβ₁₋₄₂.

6. The method of claim 1, further comprising examining the brains of the 3xTg-AD mice and the control mice by detecting β-amyloid containing plaques with at least one antibody that will detect β-amyloid containing plaques.

7. The method of claim 6, wherein the at least one antibody is selected from the group consisting of anti-Aβ 6E10, anti-Aβ 4G8, anti-Aβ 1560, anti-Aβ A11, anti-APP 22C11, anti-Tau HT7, anti-Tau AT8, anti-Tau AT180, anti-Tau C17, anti-Tau 5, anti-GFAP, and anti-actin.

8. The method of claim 1, further comprising isolating RNA from at least one of representative hippocampal and frontal cortical regions of the brains of the 3xTg-AD mice and the brains of the control mice.

9. The method of claim 8, further comprising hybridizing the isolated RNA to nucleic acid microarrays comprising probes for a plurality of genes of the mouse genome, and detecting the hybridization of the isolated RNA to the nucleic acid microarrays, whereby the identity of candidate biomarkers comprise genes differentially expressed in the 3xTg-AD mice and the control mice.

10. The method of claim 1, further comprising isolating protein from at least one of representative hippocampal and frontal cortical regions of the brains of the 3xTg-AD mice and the brains of the control mice.

11. The method of claim 10, further comprising analyzing the proteins using at least one of two-dimensional electrophoresis and mass spectrometry, whereby the identity of candidate biomarker proteins differentially expressed in the 3xTg-AD and the control mice is determined.

12. The method of claim 1, further comprising developing treatments for individuals suffering from AD within the human population.

13. The method of claim 1, further comprising at least one of:
at each time point, quantifying the levels of at least one of β-amyloid containing plaques, intracellular neurofibrillary tangles, and abnormally phosphorylated tau protein;
examining the brains of the 3xTg-AD mice and the control mice by detecting β-amyloid containing plaques with at least one antibody that will detect β-amyloid containing plaques;
isolating RNA from at least one of representative hippocampal and frontal cortical regions of the brains of the 3xTg-AD mice and the brains of the control mice, wherein the isolated RNA is hybridized to nucleic acid microarrays comprising probes for a plurality of genes of the mouse genome, and detecting the hybridization of the isolated RNA to the nucleic acid microarrays, whereby the identity of candidate biomarkers comprise genes differentially expressed in the 3xTg-AD mice and the control mice;
comprising isolating protein from at least one of representative hippocampal and frontal cortical regions of the brains of the 3xTg-AD mice and the brains of the control mice; and
developing treatments for individuals suffering from AD within the human population.

14. The method of claim 13, wherein Aβ₁₋₄₀ and Aβ₁₋₄₂ is quantified in the 3xTg-AD mice and the control mice.

15. The method of claim 13, further comprising analyzing the proteins using at least one of two-dimensional electrophoresis and mass spectrometry, whereby the identity of candidate biomarker proteins differentially expressed in the 3xTg-AD and the control mice is determined.

16. The method of claim 13, wherein the at least one antibody is selected from the group consisting of anti-Aβ 6E10, anti-Aβ 4G8, anti-Aβ 1560, anti-Aβ A11, anti-APP 22C11, anti-Tau HT7, anti-Tau AT8, anti-Tau AT180, anti-Tau C17, anti-Tau 5, anti-GFAP, and anti-actin.

17. An improved process for discovering, mining, and otherwise addressing indicia selected from the group consisting of genes, proteins, metabolites, and related biomarkers associated with neurodegenerative disease states comprising, in combination:
identifying aspects of disease to be investigated;
assaying aspects of the disease to be investigated to generate a data set of aspects of the disease;
comparing the data set of aspects of the disease against a control data set to determine a candidate set of biomarkers; and
providing indicators for responses of animals that model response to at least one of specified therapeutics, dosages, and treatment regimens.

18. The process of claim 17, wherein the indicator are bioconjugated quantum dot nanocrystals, linked to biological molecules and capable of stable fluorescent light emission and multiplexing.

19. A product by the process of claim 18.

20. The process of claim 17, wherein the aspects of the disease comprise at least one of signature biochemical networks, deposits of β-amyloid protein, neurofibrillary tangles, abnormally phosphorylated tau protein.

21. A nanosytems biological approach to development of clinical diagnostic tools for treating neurodegenerative disease comprising, in combination:
utilizing a gene expression profiling protocol in conjunction with phenotypic analysis to understand subject gene expression patterns and neuroanatomical alterations;
measuring protein expression levels to identify protein-protein interactions of identified candidates;
performing immunohistochemical, gene, and protein expression analyses in conjunction with monitoring progression and pathogenesis behaviorally;
functionally analyzing expressed data to discover novel molecular networks in comparison to user-defined lists to known biological association networks databases; and
introducing new molecular diagnostic assays for accurate, predictive, and early and pre-symptomatic detection of neurodegenerative disease in the human population.
